(19) United States
(12) Patent Application Publication
(10) Pub. No.: US 2023/0372086 A1
(43) Pub. Date: Nov. 23, 2023

(54) SYSTEMS AND METHODS PREVENTING PARAVALVULAR LEAK (PVL) ASSOCIATED WITH A REPLACEMENT STRUCTURAL CARDIAC VALVE

(71) Applicant: VALCARE, INC., Herzelyia Pituach (IL)

(72) Inventors: Aviad Galler, Tel aviv (IL); Nadav Yellin, Aven Yehuda (IL); Orit Hameiri, Kefar Saba (IL); Shuki Porath, Haifa (IL); Yoav Rozen, Binyamina (IL)

(21) Appl. No.: 18/321,240

(22) Filed: May 22, 2023

Related U.S. Application Data
(60) Provisional application No. 63/365,149, filed on May 23, 2022.

(51) Int. Cl. A61F 2/24

(52) U.S. Cl. CPC A61F 2/2409; A61F 2/2448; A61F 2250/0063; A61F 2220/0008; A61F 2230/0078; A61F 2220/0025; A61F 2250/0096; A61F 2250/0067; A61F 2002/0086

(57) ABSTRACT

The present disclosure relates to system and methods for preventing paravalvular leak (PVL) associated with a replacement of a structural cardiac valve. Specifically the disclosure relates to system and methods operable to prevent PVL by restricting the axial translation of a resilient prosthetic valve scaffold within a containing ring.

### RELATED CASES

This application claims priority to U.S. Provisional Application No. 63/365,149, filed on May 23, 2022, and titled “SYSTEMS AND METHODS PREVENTING PARAVALVULAR LEAK (PVL) ASSOCIATED WITH A REPLACEMENT STRUCTURAL CARDIAC VALVE,” which is hereby incorporated by reference in its entirety.

### BACKGROUND

The present disclosure is directed to system and methods for preventing paravalvular leak associated with a replacement structural cardiac valve. Specifically the disclosure is directed to systems and methods operable to prevent the axial translation of a resilient prosthetic valve scaffold within a containing ring and of the containing ring within the annulus.

Paravalvular leak (PVL) is a common and challenging problem that has existed since prosthetic cardiac valves were first implanted. PVL typically occurs in 7% to 17% of mitral valve replacements (MVRs) and 5% to 10% of aortic valve replacements (AVRs), typically associated with various disabling symptoms related to heart failure or hemolysis, such as, for example increased risks for endocarditis and hemolytic anemia.

Transcatheter aortic valve implantation (TAVI), and transcatheter mitral valve implantation (TMVI) are both emerging minimally invasive treatment modalities that has been shown to reduce mortality significantly compared to medical therapy alone, and are superior to conventional surgical aortic/mitral valve replacement (SA/MVR) in patients with severe and symptomatic valvular stenosis deemed to have an intermediate or higher operative risk. Paravalvular leak (PVL) is common after TAVI, associated with worse survival. Generally, PVL severity is thought to be worst at the time of implantation and improve over time, with incidence of post-TAVI, PVL reported to be more frequent than after SAVR and, more importantly PVL was shown to be associated with poor long-term procedural outcome, kidney injury and life-threatening bleeding. Even mild PVL has demonstrated limiting long-term prognosis.

Repeat surgery to repair PVL is associated with significant mortality and morbidity. Tissues around PVL are often calcified and friable, causing leaks to recur. Conservative management of mild to moderate leaks is also associated with high adverse event rates because leaks tend to deteriorate over time.

The proposed systems, methods and compositions aim to address the above-identified shortcomings.

### SUMMARY

In an exemplary implementation, provided herein is system for preventing paravalvular leak (PVL) associated with a replacement structural cardiac valve, comprising: a ring, operably coupled to a tissue annulus of a structural cardiac valve, the ring operable to actively couple the tissue to the ring, and prevent axial translation of the ring relative to the annulus; and a resilient prosthetic valve scaffold, enclosed within, and coupled to the ring operable to engage both the ring and a portion of the structural cardiac valve, wherein the system is operable to prevent axial translation between the ring and the resilient prosthetic valve scaffold.

In another exemplary implementation, the ring comprises: a hollow tube comprising: a first end; and a second end; a mesh fabric sleeve having a plurality of openings defined therein; at least one snap mechanism configured to connect the first end and the second end together, forming a ring with a predetermined internal circumference; a plurality of anchor deployment zones; a plurality of zone-specific, strained and resilient anchors, each zone-specific, strained and resilient anchor configured to transition between insertion configuration within the ring and deployment configuration, wherein each of the zone-specific strained anchors are configured to engage the annulus tissue of the structural cardiac valve, and continuously bias the tissue toward the ring.

In yet another exemplary implementation, the resilient prosthetic valve scaffold is operable to transition from an insertion configuration to an operative configuration, whereby, in the operative configuration, the resilient prosthetic valve scaffold forms a hyperboloid of one sheet, having a basal end and an apical end defining a longitudinal axis, the ring is sized and configured to accommodate the resilient prosthetic valve scaffold.

In an exemplary implementation, provided herein is a method for preventing paravalvular leak following cardiac valve replacement, implementable in a system comprising a ring, operably coupled to a tissue annulus of a structural cardiac valve and prevent axial translation of the ring relative to the annulus, wherein the ring comprises: a hollow tube comprising: a first end; and a second end; a mesh fabric sleeve having a plurality of openings defined therein; at least one snap mechanism configured to connect the first end and the second end to each other, forming a ring with a predetermined internal circumference; a plurality of anchor deployment zones; a plurality of zone-specific, strained and resilient anchors, each zone-specific, strained and resilient anchor configured to transition between insertion configuration within the ring and deployment configuration, wherein each of the zone-specific strained anchors are configured to engage the annulus tissue of the structural cardiac valve, and continuously bias the tissue toward the ring and prevent axial translation of the ring relative to the annulus; and a resilient prosthetic valve scaffold, enclosed within, and coupled to the ring operable to engage both the ring and a portion of the structural cardiac valve, the resilient prosthetic valve scaffold is operable to transition from an insertion configuration to an operative configuration, whereby, in the operative configuration, the resilient prosthetic valve scaffold forms a hyperboloid of one sheet, having a basal end and an apical end defining a longitudinal axis with a plurality of resilient clamps, wherein the ring is sized and configured to accommodate the resilient prosthetic valve scaffold, the system is operable to prevent axial translation between the ring and the resilient prosthetic valve scaffold, the method comprising: using a first delivery catheter included with the system, operable to deploy the ring, engaging the cardiac valve's annulus with the zone-specific, strained and resilient anchors at a predetermined orientation; and using a second delivery catheter included with the system, transitioning the resilient prosthetic valve scaffold from the insertion configuration to the operable configuration within the internal periphery of the ring, such that the ring encloses the resilient prosthetic valve scaffold's hyperboloid vertex.

### BRIEF DESCRIPTION OF THE DRAWINGS

The method implementable using the alignment and engagement systems disclosed herein will become apparent from the following detailed description when read in conjunction with the figures, which are exemplary, not limiting, and in which:

FIG. 1, illustrates an exemplary implementation of the resilient prosthetic valve scaffold engaged within the ring;

FIG. 2, illustrates the resilient prosthetic valve scaffold in an insertion configuration;

FIGS. 3A, illustrates a perspective view of the resilient prosthetic valve scaffold in its operational configuration, with FIG. 3B illustrating a top plan view thereof;

FIGS. 4A, illustrates the clamps of resilient prosthetic valve scaffold in insertion configuration, with FIG. 4B, illustrating the clamps in operational configuration;

FIGS. 5A, illustrates the spurs of resilient prosthetic valve scaffold in insertion configuration, with FIG. 5B, illustrating the spurs in operational configuration;

FIGS. 6A, illustrates the vertex (waist) apertures of resilient prosthetic valve scaffold in insertion configuration, with FIG. 6B, illustrating the apertures in operational configuration;

FIG. 7 is a schematic illustration of an exemplary implementation of the (annuloplasty) ring;

FIG. 8, is a schematic illustration of a top plane view an exemplary implementation of a skirted resilient prosthetic scaffold fitted with a four-leaflet valve; and

FIG. 9, is a bottom perspective view of the skirted resilient prosthetic scaffold fitted with a four-leaflet valve illustrated in FIG. 8.

While the disclosure of the system and methods operable to prevent PVL by restricting the axial translation of a resilient prosthetic valve scaffold within a containing ring disclosed herein, is amenable to various modifications and alternative forms, specifics thereof have been shown by way of example in the drawings and will be further described in detail herein below. It should be understood, however, that the intention is not to limit the disclosure to the particular exemplary implementations described. On the contrary, the intention is to cover all modifications, equivalents, and alternatives.

### DETAILED DESCRIPTION

Provided herein are exemplary implementations of a system and methods operable to prevent PVL by restricting the axial translation of a resilient prosthetic valve scaffold within a containing ring, such as an annuloplasty ring. The structural cardiac valve can be, for example: a pulmonary valve, a mitral valve, a tricuspid valve, and an aortic valve.

As indicated, the goal of both TAVI and TMVI is to anchor the prosthesis for long-term limited (or non-existent) migration, while avoiding left ventricle outflow (LVOT) obstruction and paravalvular leak (PVL). Proper prosthesis sizing, and orientation, must work in collaboration to provide the valve leaflets incorporated within the prosthesis, assuming properly tethered, to perform satisfactorily.

TMVI poses several challenges that can exacerbate PVL. For example, the mitral annulus has an asymmetrical saddle shape, requiring different anchoring designs for different mitral regurgitation etiologies. Furthermore, under certain circumstances LVOT obstruction might occur due to retained native valve tissue, for example a cusp or leaflet, exacerbating PVL which, occurs not in insignificant number of procedures.

Accordingly and in an exemplary implementation, provided herein is a system for preventing paravalvular leak (PVL) associated with a replacement structural cardiac valve, comprising: a ring, operably coupled to a tissue annulus of a structural cardiac valve; and a resilient prosthetic valve scaffold, enclosed within, and coupled to the ring operable to engage both the ring and a portion of the structural cardiac valve, wherein the system is operable to prevent axial translation between the ring and the resilient prosthetic valve scaffold.

### Definitions

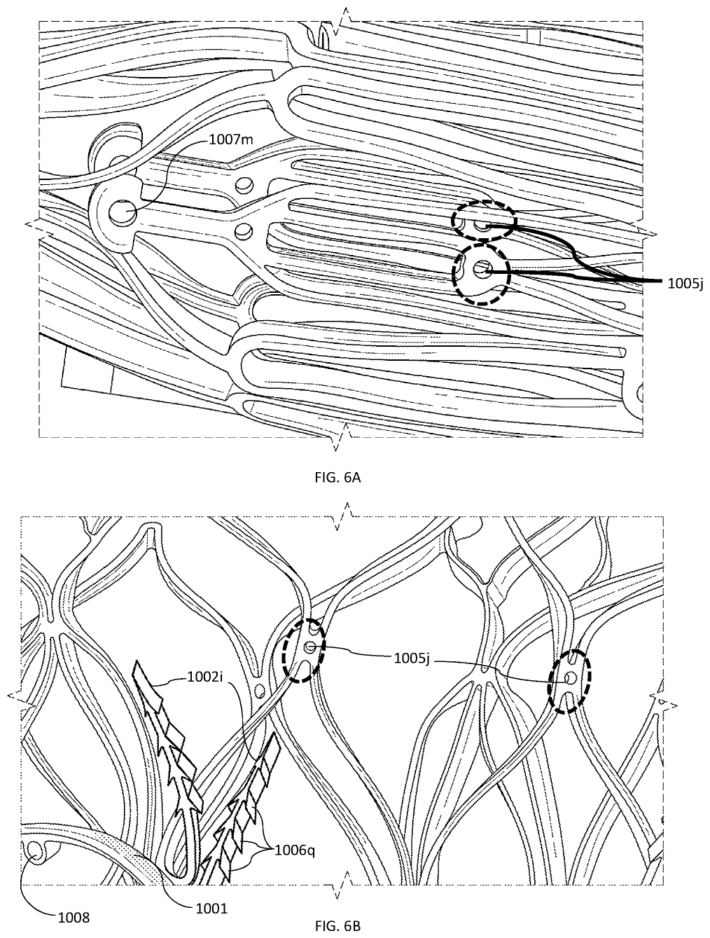

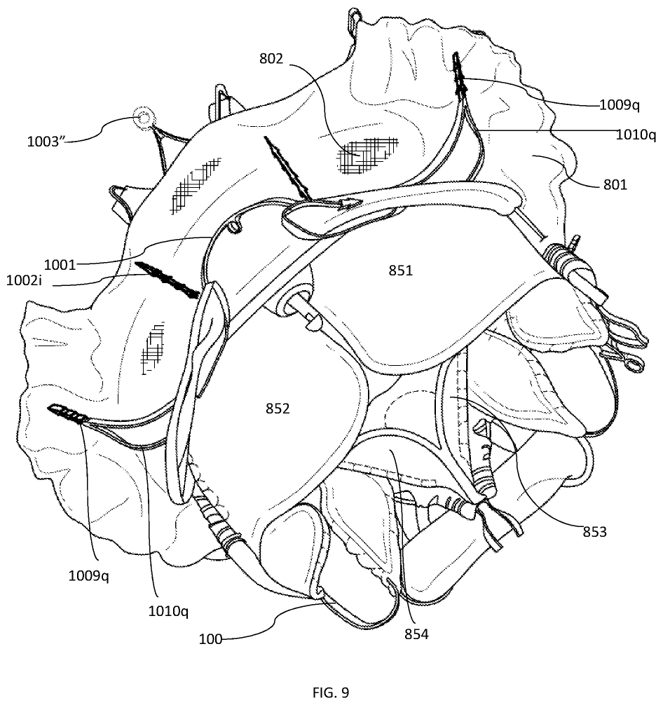

The term “scaffold” as used herein refers to a substance that provides a structural support for the structural valve itself. The plurality of cusps or leaflets, can be coupled to the resilient prosthetic valve scaffold, for example, by coupling them to apertures 1005j (see e.g., FIGS. 6B, 8 and 9), or directly to a fabric skirt covering the scaffold (see e.g., FIGS. 8, 9). The scaffold may be infused with, coated with, impregnated with, or comprised (stem) cells, growth factors, extracellular matrix components, nutrients, integrins, or other substances to promote neo-cradium tissue ingrowth. The scaffold can also be encased in a fabric skirt, forming a continuous surface either on the inside perimeter of the scaffold, the outside perimeter, or both.

In the context of the disclosure, the term “resilient prosthetic valve scaffold” refers to the ability of the prosthetic valve scaffold to readily deform upon the application of pressure, as well as its ability to generally spring back to its original shape when such pressure is removed. Moreover, the term resilient, in the context of the disclosure means that so long as the resilient prosthetic valve scaffold has not returned to its unconstrained original shape, it will exert a biasing force in the direction of the applied pressure constraining the resilient prosthetic valve scaffold.

In the context of the disclosure, the term “strained and resilient anchors” means that the anchors are operable to exert a biasing force, continuously urging the structural valve annulus or other tissue engaged by the strained and resilient anchors towards the ring surface. This is achieved in an exemplary implementation, by controlling the difference in the deployment radii and the anchors' hooks radii, such that following the transition to the deployment configuration, the strained and resilient anchors remain in a strained configuration.

The term “coupled”, including its various forms such as “operably coupling”, “coupling” or “couplable”, refers to and comprises any direct or indirect, structural coupling, connection or attachment, or adaptation or capability for such a direct or indirect structural or operational coupling, connection or attachment, including integrally formed components and components which are coupled via or through another component or by the forming process. Indirect coupling may involve coupling through an intermediary member or adhesive, or abutting and otherwise resting against, whether frictionally or by separate means without any physical connection.

In addition, for the purposes of the present disclosure, directional or positional terms such as “top”, “bottom”, “upper,” “lower,” “side,” “front,” “frontal,” “forward,” “rear,” “rearward,” “back,” “trailing,” “above,” “below,” “left,” “right,” “radial,” “vertical,” “upward,” “downward,” “outer,” “inner,” “exterior,” “interior,” “intermediate,”, “apical”, “basal”, etc., are merely used for convenience in describing the various exemplary implementations of the present disclosure.

Likewise, the term “engage” and various forms thereof, when used with reference to an engaging element, for example in the engagement of at least one native leaflet, or cusp by clamp 1001, refers in an exemplary implementation to the application of any forces that tend to hold at least one clamp 1001 and at least one native valve cusp, and/or valve leaflet together against inadvertent or undesired separating forces (e.g., such as may be introduced during the pulsatile blood flow through the replaced structural cardiac valve). It is to be understood, however, that engagement does not in all cases require an interlocking connection that is maintained against every conceivable type or magnitude of separating force. Further, the term “engaging element” refers in another exemplary implementation to one or a plurality of coupled components, at least one of which is configured for releasably engaging another element. Thus, this term encompasses both single part engaging elements and multi-part-assemblies. For example, clamp 1001 can be interchangeably referred to as “engaging member”, or “engaging element”. Accordingly, in the context of the disclosure, the term “clamp” means a member of the resilient prosthetic valve scaffold operable to engage a native leaflet or cusp of the structural heart valve sought to be replaced, regardless if that structural heart valve is a mitral valve (MV), tricuspid valve (TV), aortic valve (AV), or the pulmonary valve (PV).

The terms “first,” “second,” and the like, herein do not denote any order, quantity, or importance, but rather are used to denote one element from another. The terms “a”, “an” and “the” herein do not denote a limitation of quantity, and are to be construed to cover both the singular and the plural, unless otherwise indicated herein or clearly contradicted by context. The suffix “(s)” as used herein is intended to include both the singular and the plural of the term that it modifies, thereby including one or more of that term (e.g., anchor(s) 2022p includes one or more anchor).

Reference throughout the specification to “one exemplary implementation”, “another exemplary implementation”, “an exemplary implementation”, and so forth, means that a particular element (e.g., step, feature, structure, and/or characteristic) described in connection with the exemplary implementation is included in at least one exemplary implementation described herein, and may or may not be present in other exemplary implementations. In addition, it is to be understood that the described elements may be combined in any suitable manner in the various exemplary implementations.

In the context of the disclosure, the term “operable” means the system and/or the device, or a certain element or step is fully functional, sized, adapted and calibrated, comprises elements for, and meets applicable operability requirements to perform a recited function when activated, coupled, implemented, actuated, effected, or realized. In relation to systems, the term “operable” means the system is fully functional and calibrated, having the necessary elements, as well as the mechanisms for, and meets applicable operability requirements to perform a recited function when executed by a user.

In the context of the disclosure, the term “saddle-shaped” is used herein to mean an annuloplasty ring generally made of two arcuate members for example, two toroidal portions with each toroidal portion having an apex and two ends connecting the toroidal portions. The apex of the toroidal portion in one member can be in the same or opposite direction of the other member. The formed ring can be generally D-shaped.

A more complete understanding of the systems and methods operable to prevent the axial translation of a resilient prosthetic valve scaffold within a containing ring, can be obtained by reference to the accompanying drawings. These figures (also referred to herein as “FIG.”) are merely schematic representations based on convenience and the ease of demonstrating the present disclosure, and are, therefore, not intended to indicate relative size, scale and dimensions of the devices or components thereof, and/or to define or limit the scope of the exemplary implementations. Although specific terms are used in the following description for the sake of clarity, these terms are intended to refer only to the particular structure of the exemplary implementations selected for illustration in the drawings, and are not intended to define or limit the scope of the disclosure. In the drawings and the following description below, it is to be understood that like numeric designations refer to components of like function.

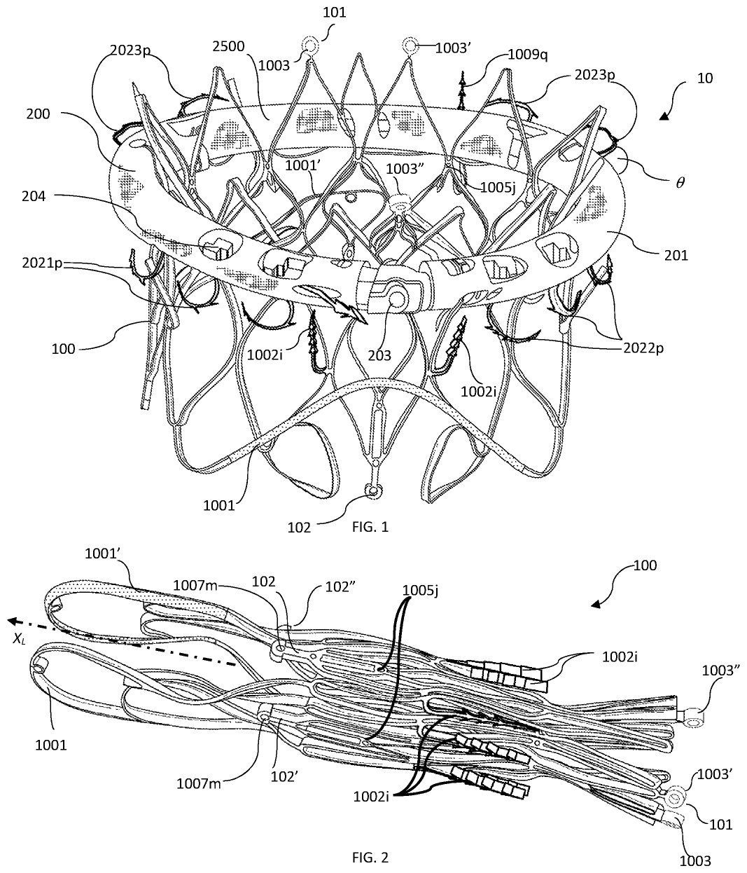

Turning now to FIGS. 1-3B and 7, illustrating an exemplary implementation of the system for preventing PVL. As illustrated, system 10 for preventing PVL associated with a replacement structural cardiac valve (e.g., mitral, tricuspid, aortal or pulmonary valves), comprises: ring 200, operably coupled to a tissue annulus of the structural cardiac valve, ring 200 operable to actively couple the tissue to ring 200 (not shown); and resilient prosthetic valve scaffold 100, enclosed within, and coupled to ring 200, prosthetic valve scaffold 100 being operable to engage both ring 200 and a portion of the structural cardiac valve such as a tissue, a wall or a portion of the valve such as a cusp or a leaflet, wherein system 10 is operable to prevent axial translation between ring 200 and resilient prosthetic valve scaffold 100.

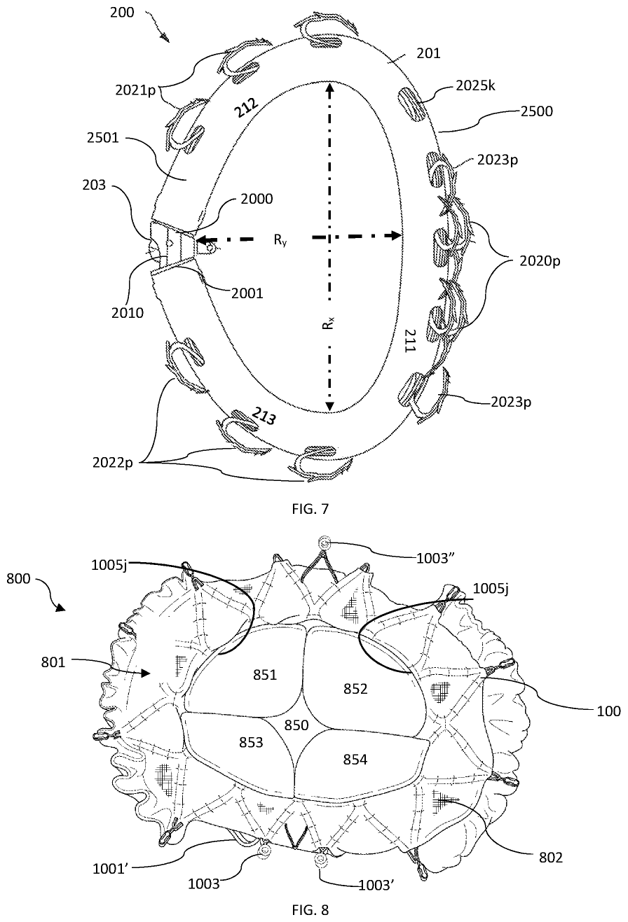

Turning now to FIGS. 1, and 7, illustrating an exemplary implementation of ring 200, which can be an annuloplasty ring. As illustrated, ring 200 having apical end surface 2500 (see e.g., FIG. 1, and basal surface 2501 (see e.g., FIG. 7), Ring 200 further comprises hollow tube 201 comprising: first end 2000; and second end 2001; mesh fabric sleeve 201 having plurality of openings 2025k defined therein; at least one snap mechanism 203 configured to connect first end 2000 and second end 2001 together, forming ring with predetermined internal circumference (it is noted that the circumference does not necessarily needs to be circular and can be interchangeable with diameter).

Also illustrated in FIGS. 1, and 7 are plurality of anchor deployment zones 211, 212, 213, having plurality of zone-specific, strained and resilient anchors 2020p, 2021p, 2022p, 2023p, each p^{th} zone-specific, strained and resilient anchor 2020p, 2021p, 2022p, 2023p, configured to transition between insertion configuration within ring and deployment configuration, wherein each p^{th} zone-specific strained anchors 2020p, 2021p, 2022p, 2023p, configured to engage annulus tissue of structural cardiac valve, and continuously bias tissue toward ring, thereby actively inhibiting PVL between the annulus and ring 200 periphery. Additionally, s illustrated in FIGS. 1, and 7, zone-specific, strained and resilient anchor(s) 2020p, 2021p, 2022p, 2023p, are angled (see e.g., θ, FIG. 1), sized, adapted and configured additionally to prevent axial movement of ring 200 relative to the structural heart valve sought to be replaced. Pulsatile flow among the various hear chambers can create inconsistent stress rate on the ring and may, in certain cases, dislodge the ring from the annulus. Having anchor(s), for example 2202p curved upward is, in an exemplary implementation, beneficial for further securing ring 200 to the annulus of the heart valve sought to be replaced. This is in addition to the strain under which zone-specific, strained and resilient anchor 2020p, 2021p, 2022p, 2023p, are kept under, configured to continuously bias the annulus toward ring 200.

Ring 200 is adapted to be delivered through a catheter depending on the structural cardiac valve sought to be replaced. In an exemplary implementation, ring 200 is operable to stabilize the annulus of the structural cardiac valve sought to be replaced, reduce radial dimension of the native valve, actively engage the annulus biasing the tissue continuously toward the ring periphery, and provide the anchor for prosthetic valve scaffold 100. During the delivery from the catheter, the system changes its geometrical configuration from a linear tubular system to a close ring, with a shape sized and configured to compliment the internal shape of the valve's annulus both in terms of axial dimension (e.g., D-shape, ovoid, circular), as well as planar dimension (e.g., saddle shape). After the deployment in the atrium is completed, the implant is anchored to the native annulus using zonal 211, 212, 213 mechanism (or even more zones) using zone-specific strained anchors 2020p, 2021p, 2022p, 2023p.

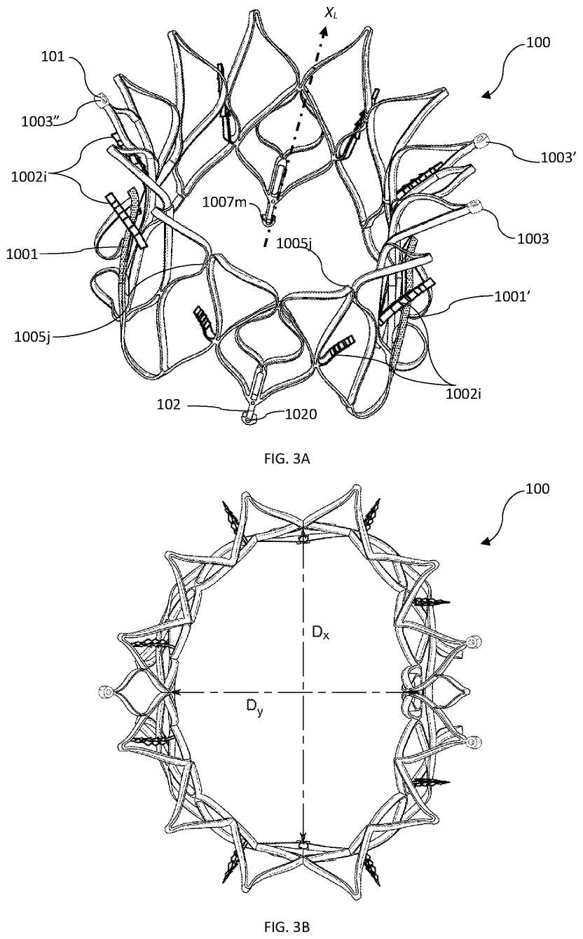

Likewise, resilient prosthetic valve scaffold 100 is operable to transition from an insertion configuration (see e.g., FIG. 2) to an operative configuration (see e.g., FIG. 3A). As indicated, once ring 200 is anchored to the annulus of the structural cardiac valve sought to be replaced, using a catheter, resilient prosthetic valve scaffold 100 is constrained within a catheter (not shown) operable to accommodate and deploy resilient prosthetic valve scaffold 100, transitioning it from its insertion configuration as illustrated in FIGS. 2, 4A, 5A, and 6A, to its operational configuration as illustrated in FIGS. 3A, 4B, 5B, and 6B.

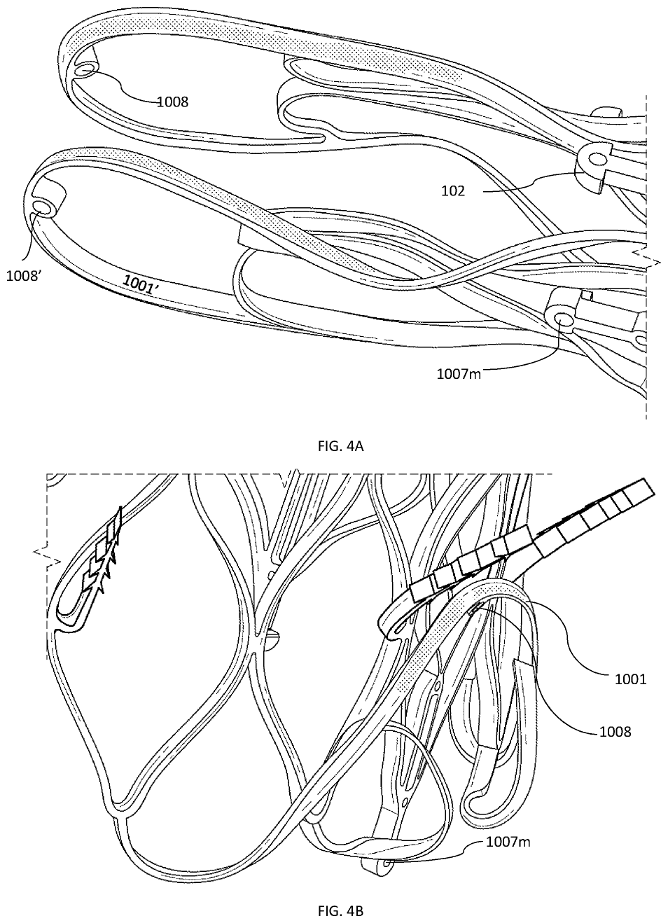

As illustrated in FIGS. 1, and 3A, in its operative configuration resilient prosthetic valve scaffold 100 forms a hyperboloid of one sheet, having basal end 102 and apical end 101 defining a longitudinal axis X_{L} (see e.g., FIG. 3A), wherein ring 200 is sized and configured to accommodate resilient prosthetic valve scaffold 100. Turning to FIGS. 1, 4A, and 4B, resilient prosthetic valve scaffold 100 further comprises plurality of resilient clamps 1001, 1001′, operable, upon transition from the insertion configuration to the operative configuration, to selectably (in other words, without affecting the operation of other components) engage at least one native leaf, or cusp of the structural cardiac valve. It is noted, that the number of clamp 1001, 1001′ can be commensurate with the cardiac valve sought to be replaced. For example, the structural cardiac valve is a mitral valve (MV), and resilient prosthetic valve scaffold 100 comprises resilient anterior clamp 1001 and resilient posterior clamp 1001′, resilient anterior clamp 1001 sized to engage native anterior leaf of the MV, while resilient posterior resilient clamp 1001′ is configured to engage native posterior leaf of the MV. In other words, the dimensions of each clamp 1001, 1001′ do not need to be equal, but can be sized for their intended engaged leaflet, or cusp. Accordingly and in another exemplary implementation, the structural cardiac valve is a tricuspid valve (TV), and wherein resilient prosthetic valve 100 scaffold comprises resilient anterior clamp 1001, resilient septal clamp 1001′, and resilient posterior clamp 1001″, resilient anterior clamp 1001 is sized to engage native anterior cusp of the TV, while resilient septal clamp 1001′ being sized to engage native septal cusp of the TV, and resilient posterior clamp 1001″ is sized to engage posterior cusp of the TV. Similarly, the structural cardiac valve is aortic valve (AV), and resilient prosthetic valve scaffold 100 comprises first resilient clamp 1001, second resilient clamp 1001′, and third resilient clamp 1001″. First resilient clamp 1001 is sized to engage native right coronary cusp (RCC) of the AV, while second resilient clamp 1001′ is sized to engage native left coronary (LCC) cusp of the AV, and third resilient clamp 1001″ is sized to engage native non-coronary cusp (NCC) of the aortic valve. Engaging the specific leaflets or cusps is beneficial in preventing at least one of: axial migration, LVOT obstruction, and PVL by further anchoring the system to the native tissue.

As illustrated, in an exemplary implementation, as shown in FIG. 3A, in its operational configuration, while FIG. 2 is in the insertion configuration, plurality of resilient clamps 1001, 1001′ are strained within the second delivery catheter, and form a parabolic arc defining eyelet 1008, 1008′ at the apogee of the arc. In an exemplary implementation, basal fiducials 102 102′, 102″, each defining eyelet (aperture) 1007m are coupled to the second delivery catheter (not shown), which is, in addition operable to control the transition of clamp 1001, 1001′ from their strained position to their unstrained position, engaging their designated leaflet for example.

In the context of the disclosure, the term “strained” is used to describe strained clamps 1001, 1001′ (and 1001″ when present), means that each of clamps 1001, 1001′ is maintained under an extrinsic tensile strain. An “extrinsic strain” as used herein refers to a tensile strain that is applied to each of clamps 1001, 1001′ by the catheter (or an external force), rather than a tensile strain developed within the strained each of clamps 1001, 1001′. In other words, in the unstrained position, each of clamps 1001, 1001′, formed of a resilient material (e.g., nitinol, stainless steel and the like) curls, and the insertion into a catheter cannula causes the tensile stress to form the strain imposed on each of clamps 1001, 1001′.

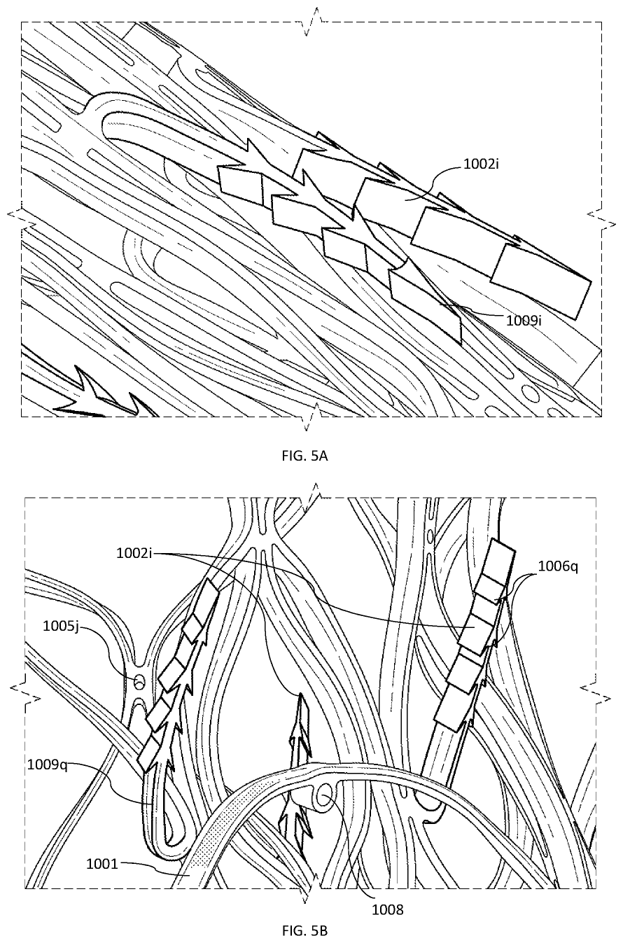

Accordingly, and in another exemplary implementation, resilient prosthetic valve scaffold 100 further comprises plurality of spurs 1002i, 1009q each i^{th} and q^{th} spur extending in the apical direction, plurality of spurs 1002i operable to engage portion of the structural cardiac valve (e.g., AV, MV, TV) and since most of the forced flow is from ventricle to the atrium, the pulsatile flow urges the spurs, into the tissue with plurality of oppositely directed burs 1006q, (see e.g., FIGS. 5A, 5B, and 6B), extending laterally from each i^{th} spur 1002i, operable to prevent the migration of each i^{th} spur 1002i, from the tissue, again making the coupling of the system to the native valve more intimate, thus acting to prevent PVL. Additionally, plurality of spurs 1009q are operable to engage at least a portion of ring 200, and as illustrated in FIG. 9, are likewise disposed on a strained extension 1010q.

Moreover, resilient prosthetic valve scaffold 100, further comprises plurality of apical fiducials 1003, 1003′, 1003″, configured to orient resilient prosthetic scaffold 100 within ring 200, relative to the structural cardiac valve. For example, the structural cardiac valve is MV and resilient prosthetic valve scaffold comprising pair of spaced apart anterior fiducials 1003, 1003′ and single posterior fiducial 1003″, configured to orient resilient prosthetic scaffold 100 within ring 200 such that pair of spaced apart anterior fiducials 1003, 1003′ are adjacent the natural fiducials of the left trigon and the right trigon, thus ensuring alignment between clamps 1001 and 1001′ and native anterior and posterior leaflets. Accordingly, in the methods disclosed, implementable in the systems disclosed, the method comprises, prior to step of engaging at least one native leaflet of the structural cardiac valve with clamps 1001, 1001′ (and 1001″ if present), using the plurality of fiducials 1003, 1003′ and 1003″ to orient resilient prosthetic scaffold 100 within ring 200 toward predetermined locations such as the natural fiducials of the left and right trigons on the cardiac valve.

In positioning resilient prosthetic scaffold 100 within ring 200, resilient prosthetic valve scaffold 100 is accommodated within ring at the vertex (in other words, the waist) of the hyperboloid of one sheet, such that ring 200 encloses resilient prosthetic valve scaffold's 100 hyperboloid vertex (See e.g., FIG. 1). Moreover, in the operable configuration, external circumference of the resilient prosthetic valve scaffold 100 at the vertex of is larger than the internal circumference of ring 200, resilient prosthetic valve scaffold 100 configured to bias ring 200 radially within the annulus structural cardiac valve, thus also preventing the migration of resilient prosthetic valve scaffold 100 within ring 200, as well as urging ring 200 to the annulus, improving the connection to the structural cardiac valve and preventing PVL. It is noted, that in certain exemplary implementations, the hyperboloid of one sheet is asymmetric, meaning the longitudinal axis of the apical end is shorter than the longitudinal axis of the basal end.

Moreover, it is noted the revolving hyperbole forming the Y-Z (J) cross section does not necessarily need to be deep, in other words, the distance between the focus and the vertex may vary, such that the angle defined between the asymptotes of the hyperbole can be between 90° and about 175°.

Moreover and in certain exemplary implementations as illustrated in FIGS. 3B, and 7-9, ring 200 can have a D-shape having a major axis R_{Y} and a minor axis R_{X}; and wherein, in the operable configuration, resilient prosthetic valve scaffold 100 has a cross section (see e.g., FIG. 3B), transverse to the longitudinal axis having a D-shape, defining major axis D_{X} that is longer than major axis Rₓ, of the D-shaped ring 200 (see e.g., FIG. 7) and minor axis D_{Y} that is larger than minor axis R_{y} of D-shaped ring 200, such that resilient prosthetic valve scaffold 100 configured to bias ring 200 radially.

Furthermore, and in certain exemplary implementations, mesh fabric sleeve 201 on ring 200, can comprise woven fabric with a thread density (in other words, number of threads per unit area, for example, between 10 and 100 threads per cm²), configured to allow tissue ingrowth of portion of the structural cardiac valve into the ring. Additionally (see e.g., FIGS. 8, 9), resilient prosthetic valve scaffold 100 is covered with mesh fabric 802 forming a continuous skirt 801, continuous skirt 801 comprises the woven fabric with the thickness of the skirt, and thread density configured to both allow tissue ingrowth of portion of the structural cardiac valve into continuous skirt 801 of resilient prosthetic valve scaffold 100, as well as further seal any gaps between resilient prosthetic valve scaffold 100 and ring 200. Similar to mesh fabric sleeve 201, woven fabric 802 of continuous skirt 801 can be impregnated with an agent configured to promote neo-endocardial tissue ingrowth into the skirt, and further promote the seal between resilient prosthetic valve scaffold 100 and ring 200.

The fabric can be any appropriate biocompatible fabric, for example, braided PET fabric, configured to promote the tissue ingrowth post implantation. Furthermore, the fabric mesh can further be coated, or impregnated with an agent configured to promote neo-endocardial tissue ingrowth into the mesh fabric sleeve, such as for example, at least one of insulin-like growth factor 1 (IGF-1), neuregulin, and platelet-derived growth factor (PDGF), [which others?] or a composition comprising the foregoing. Likewise, a similar sleeve can be used to cover the exterior of resilient prosthetic valve scaffold 100.

Turning now to FIGS. 8, and 9, illustrating resilient prosthetic valve scaffold 100 covered with the mesh fabric 802 forming continuous skirt 801, continuous skirt 801 comprises woven fabric with a thread density configured to allow tissue ingrowth of portion of the structural cardiac valve into skirt 801 of resilient prosthetic valve scaffold 100. As illustrated, cardiac replacement valve 800 further comprising plurality of leaflets 850 operably coupled to skirt 801, whereby leaflets 850 operable as replacement structural cardiac replacement valve 800. In an exemplary implementation, cardiac replacement valve 800 comprises four leaflets two posterior leaflets 851, 852, and two anterior valves 853, 854 operable as replacement for a mitral valve. It is noted that the shape of the two posterior leaflets 851, 852, can be sized and configured to emulate the native posterior leaflet, while the two anterior leaflets 853, 854 can be sized and configured to emulate the native anterior leaflet of the replaced mitral valve. In certain other exemplary implementations, the leaflets (or cusps, depending on the structural heart valve sought to be replaced, will have the same size. In an exemplary implementation, cardiac replacement valve 800, is incorporated within ring 200 and used to replace mitral valve.

In an exemplary implementation, the methods disclosed are implementable with the systems described. Accordingly, and in one exemplary implementation, provided herein is a method for preventing paravalvular leak following cardiac valve replacement, implementable in a system comprising a ring, operably coupled to a tissue annulus of a structural cardiac valve, wherein the ring comprises: a hollow tube comprising: a first end; and a second end; a mesh fabric sleeve having a plurality of openings defined therein; at least one snap mechanism configured to connect the first end and the second end together (in other words, to each other), forming a ring with a predetermined internal circumference (or internal diameter if circular); a plurality of anchor deployment zones; a plurality of zone-specific, strained and resilient anchors, each zone-specific, strained and resilient anchor configured to transition between insertion configuration within the ring and deployment configuration, wherein each of the zone-specific strained anchors are configured to engage the annulus tissue of the structural cardiac valve, prevent axial translation of the ring relative to the annulus, and continuously bias the tissue toward the ring; and a resilient prosthetic valve scaffold, enclosed within, and coupled to the ring operable to engage both the ring and a portion of the structural cardiac valve, the resilient prosthetic valve scaffold is operable to transition from an insertion configuration to an operative configuration, whereby, in the operative configuration, the resilient prosthetic valve scaffold forms a hyperboloid of one sheet, having a basal end and an apical end defining a longitudinal axis with a plurality of resilient clamps, wherein the ring is sized and configured to accommodate the resilient prosthetic valve scaffold, the system is operable to prevent axial translation between the ring and the resilient prosthetic valve scaffold, the method comprising: using a first delivery catheter included with the system, operable to deploy the ring, engaging the cardiac valve's annulus with the zone-specific, strained and resilient anchors at a predetermined orientation; and using a second delivery catheter included with the system, transitioning the resilient prosthetic valve scaffold from the insertion configuration to the operable configuration within the internal periphery of the ring, such that the ring encloses the resilient prosthetic valve scaffold's hyperboloid vertex.

In addition, resilient prosthetic valve scaffold 100 further comprises plurality of resilient clamps 1001, 1001′ (with 1001″ [not shown] in other exemplary implementations) operable, upon transition from the insertion configuration to the operative configuration, to engage at least one of: a native leaflet, and a native cusp of the structural cardiac valve, the method further comprising using plurality of clamps 1001, 1001′ (with 1001″ for tricuspid and aortal valve replacement), engaging at least one of: at least one native leaflet, and at least one cusp of the structural cardiac valve.

Moreover, as resilient prosthetic valve scaffold 100 further comprises plurality of apical fiducials 1003, 1003′, and 1003″ configured to orient resilient prosthetic scaffold 100 within ring 200, relative to the structural cardiac valve, the method further comprising, prior to the step of engaging at least one native leaf of the structural cardiac valve, using plurality of apical fiducials 1003, 1003′, and 1003″—orienting resilient prosthetic scaffold 100 within ring 200 toward predetermined locations on the cardiac valve. As illustrated in FIGS. 4B and 6A, resilient prosthetic valve scaffold 100 further comprises plurality of basal fiducials 102, 102′, 102″, each defining an aperture (interchangeable with eyelet) 1007m therein, configured to couple resilient prosthetic valve scaffold 100 to second delivery catheter (the first is configured to deliver the ring and transition the ring from insertion configuration to an operational, deployed configuration and operably couple the ring to the annulus of the structural cardiac valve sought to be replaced) and assist in the deployment and orientation of resilient prosthetic valve scaffold 100 within ring 200 and the annulus of the structural cardiac valve sought to be replaced.

Also, resilient prosthetic valve scaffold, further comprises plurality of spurs 1002i, each spur extending in apical 101 direction (see e.g., FIG. 1), with plurality of spurs 1002i operable to engage the portion of the structural cardiac valve, and the method further comprising, prior to the step of engaging at least one native leaf of the structural cardiac valve, using plurality of spurs 1002i, engaging the portion of the structural cardiac valve. Similarly, resilient prosthetic valve scaffold 100 further comprises plurality of apertures 1005j defined at the hyperboloid vertex of resilient prosthetic valve scaffold 100 (optionally providing an additional reference point for placing resilient prosthetic valve scaffold 100 within and relative to ring 200), the method further comprising, following the step of engaging at least one native leaf of the structural cardiac valve, using a suture, suturing the resilient prosthetic valve scaffold to the ring through the plurality of apertures. Additionally or alternatively, in certain exemplary implementations (see e.g., FIGS. 1, 9) plurality of spurs 1009q are operable to engage at least a portion of ring 200, and as illustrated in FIG. 9, are likewise disposed on a strained extension 1010q.

Accordingly and in an exemplary implementation, the structural cardiac valve is a mitral valve, resilient prosthetic valve scaffold 100 and comprises pair of spaced apart anterior 1003′, 1003″ fiducials and single posterior fiducial 1003, the method comprising: using pair of spaced apart anterior fiducials 1003′, 1003″, orienting resilient prosthetic scaffold 100 within ring 200 (after coupling ring 200 to mitral valve annulus) such that pair of spaced apart anterior fiducials 1003′, 1003″ are adjacent to the left trigon and the right trigon; and using resilient anterior clamp 1001′, engaging the anterior leaflet; and using posterior clamp 1001, engaging the posterior leaflet, wherein the engagement order is optionally reversed, wherein resilient prosthetic valve scaffold 100 comprises four leaflets 851, 852, 853, 854 (see e.g., FIGS. 8, 9) operable replacement for a mitral valve leaflets.

In other exemplary implementations, the leaflet are adapted, sized, shaped and configured, (for example by the (apically) concave shape, the resiliency and stiffness of the leaflet/cusps, and the shape of the leaflets/cusps) to transition between a sealed position once atrial pressure exceeds ventricle pressure, to an open position, when atrial pressure is below ventricle pressure.

It is further noted, that if the valve sought to be replaced is any one of: an aortic valve, and a pulmonary valve, the systems and methods disclosed may still be practiced and the trans-valvar pressure differentials and flow direction, as well as the leaflets/cusps structure will determine the adaptation, size, and configuration (in other words, the concave nature and their direction) of the leaflets incorporated into the resilient prosthetic valve scaffold 100. In these exemplary implementations, the terms apical and basal will not be directed necessarily to ventricle and atrium, but rather to the left ventricle (LV) (basal) and the aorta (apical), in replacement of the aortal valve, or between the right ventricle (RV) (basal) and the pulmonary artery (apical).

While in the foregoing specification the methods, systems, sub systems and kits of for preventing paravalvular leak (PVL) associated with a replacement of a structural cardiac valve, described herein have been described in relation to certain exemplary implementations, and many details are set forth for purpose of illustration, it will be apparent to those skilled in the art that the disclosure of the alignment methods, implementable using the systems disclosed herein are susceptible to additional implementations and that certain of the details described in this specification and as are more fully delineated in the following claims can be varied considerably without departing from the basic principles disclosed herein.

1. A system for preventing paravalvular leak (PVL) associated with a replacement structural cardiac valve, comprising:
a. a ring, operably coupled to a tissue annulus of a structural cardiac valve, the ring operable to actively couple the tissue to the ring, and prevent axial translation of the ring relative to the tissue annulus; and
b. a resilient prosthetic valve scaffold, enclosed within, and coupled to the ring operable to engage both the ring and a portion of the structural cardiac valve, wherein the system is operable to prevent axial translation between the ring and the resilient prosthetic valve scaffold.

2. The system of claim 1, wherein the ring comprises:
a. a hollow tube comprising:
i. a first end; and
ii. a second end;
b. a mesh fabric sleeve having a plurality of openings defined therein;
c. at least one snap mechanism configured to connect the first end and the second end together, forming a ring with a predetermined internal circumference;
d. a plurality of anchor deployment zones; and
e. a plurality of zone-specific, strained and resilient anchors, each zone-specific, strained and resilient anchor configured to transition between insertion configuration within the ring and deployment configuration, wherein each of the zone-specific strained anchors are configured to engage the annulus tissue of the structural cardiac valve, continuously bias the tissue toward the ring, and prevent axial translation of the ring relative to the annulus.

3. The system of claim 2, wherein the resilient prosthetic valve scaffold is operable to transition from an insertion configuration to an operative configuration.

4. The system of claim 3, wherein, in the operative configuration the resilient prosthetic valve scaffold forms a hyperboloid of one sheet, having a basal end and an apical end defining a longitudinal axis, wherein the ring is sized and configured to accommodate the resilient prosthetic valve scaffold.

5. The system of claim 4, wherein the resilient prosthetic valve scaffold further comprises a plurality of resilient clamps, operable, upon transition from the insertion configuration to the operative configuration, to engage at least one of: a native leaflet, and a native cusp of the structural cardiac valve.

6. The system of claim 4, wherein the resilient prosthetic valve scaffold, further comprises a plurality of apical fiducials, configured to orient the resilient prosthetic scaffold within the ring, relative to the structural cardiac valve.

7. The system of claim 4, wherein the resilient prosthetic valve scaffold, further comprises a plurality of spurs, each spur extending in the apical direction, the plurality of spurs operable to engage at least one of: the portion of the structural cardiac valve, and at least a portion of the ring.

8. The system of claim 4, wherein the resilient prosthetic valve scaffold further comprises a plurality of apertures defined at the hyperboloid vertex.

9. The system of claim 4, wherein the resilient prosthetic valve scaffold is accommodated within the ring at the vertex of the hyperboloid of one sheet.

10. The system of claim 9, wherein, in the operable configuration, the external circumference of the resilient prosthetic valve scaffold at the vertex has an external circumference that is larger than the internal circumference of the ring.

11. The system of claim 4, wherein the ring has a D-shape having a major axis and a minor axis; and wherein, in the operable configuration, the resilient prosthetic valve scaffold has a cross section, transverse to the longitudinal axis having a D-shape, defining a major axis that is longer than the major axis of the D-shaped ring and a minor axis that is larger than the minor axis of the D-shaped ring, the resilient prosthetic valve scaffold configured to bias the ring radially.

12. The system of claim 2, wherein the mesh fabric sleeve comprises a woven fabric with a thread density configured to allow ingrowth of portion of the structural cardiac valve into the ring, and provide a seal between the tissue annulus and the ring.

13. The system of claim 12, wherein the sleeve is coated with an agent configured to promote neo-endocardial tissue ingrowth into the mesh fabric sleeve.

14. The system of claim 4, wherein the structural cardiac valve is a mitral valve, and wherein the resilient prosthetic valve scaffold comprises a resilient anterior clamp and a resilient posterior clamp, the resilient anterior clamp sized to engage a native anterior leaflet of the mitral valve, and wherein the resilient posterior resilient clamp is configured to engage a native posterior leaflet of the mitral valve.

15. The system of claim 4, wherein the structural cardiac valve is a tricuspid valve, and wherein the resilient prosthetic valve scaffold comprises a resilient anterior clamp, a resilient septal clamp, and a resilient posterior clamp, the resilient anterior clamp is sized to engage to a native anterior cusp of the tricuspid valve, wherein the resilient septal clamp is sized to engage to a native septal cusp of the tricuspid valve, and wherein the resilient posterior clamp is sized to engage to a native posterior cusp of the tricuspid valve.

16. The system of claim 4, wherein the structural cardiac valve is an aortic valve, and wherein the resilient prosthetic valve scaffold comprises a first resilient clamp, a second resilient clamp, and a third resilient clamp, the first resilient clamp is sized to engage a native right coronary cusp (RCC) of the aortic valve, wherein the second resilient clamp is sized to engage a native left coronary (LCC) cusp of the aortic valve, and wherein the third resilient clamp is sized to engage a native non-coronary cusp (NCC) of the aortic valve.

17. The system of claim 6, wherein the structural cardiac valve is a mitral valve, the resilient prosthetic valve scaffold comprising a pair of spaced apart anterior fiducials and a single posterior fiducial, configured to orient the resilient prosthetic scaffold within the ring such that the pair of spaced apart anterior fiducials are adjacent the left trigon and the right trigon.

18. The system of claim 4, wherein the hyperboloid of one sheet is asymmetric, wherein the longitudinal axis of the apical end is shorter than the longitudinal axis of the basal end.

19. The system of claim 13, wherein the agent configured to promote neo-endocardial tissue ingrowth into the mesh fabric sleeve is at least one of: insulin-like growth factor 1 (IGF-1), neuregulin, and platelet-derived growth factor (PDGF), or a composition comprising the foregoing.

20. The system of claim 4, wherein the exterior of the resilient prosthetic valve scaffold is covered with the mesh fabric forming a continuous skirt, the continuous skirt comprises a woven fabric with a thread density configured to allow ingrowth of portion of the structural cardiac valve into the skirt, and provide a seal between the resilient prosthetic valve scaffold and the ring.

21. The system of claim 20, wherein the woven fabric is impregnated with an agent configured to promote neo-endocardial tissue ingrowth into the mesh fabric sleeve.

22. The system of claim 20, further comprising a plurality of leaflets operably coupled to at least one of: the apertures, and the continuous skirt, the leaflets operable as the replacement structural cardiac valve.

23. The system of claim 22, comprising four leaflets operable as replacement for a mitral valve.

24. The system of claim 21, wherein the agent configured to promote neo-endocardial tissue ingrowth into the skirt is at least one of: insulin-like growth factor 1 (IGF-1), neuregulin, and platelet-derived growth factor (PDGF), or a composition comprising the foregoing.

25. A method for preventing paravalvular leak following cardiac valve replacement, implementable in the system of claim 4, the method comprising:
a. using a first delivery catheter included with the system, operable to deploy the ring, engaging the cardiac valve's annulus with the zone-specific, strained and resilient anchors at a predetermined orientation; and
b. using a second delivery catheter included with the system, transitioning the resilient prosthetic valve scaffold from the insertion configuration to the operable configuration within the internal circumference of the ring, such that the ring encloses the resilient prosthetic valve scaffold's hyperboloid vertex.

26-42. (canceled)
